(19) United States
(12) Patent Application Publication
(10) Pub. No.: US 2003/0082294 A1
(43) Pub. Date: May 1, 2003

(54) Surface treatment method for bio-arrays

(76) Inventors: Laurakay Bruhn, Mountain View, CA (US); Geraldine F. Dellinger, Boulder, CO (US); Douglas J. Dellinger, Boulder, CO (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/001,056

(22) Filed: Oct. 31, 2001

(51) Int. Cl. A61L 2/00

(52) U.S. Cl. 427/2.1

(57) ABSTRACT

The invention provides novel methods for surface treatment of bioarrays to provide desirable substrate surface characteristics, and novel methods for fabrication of bioarrays. The present invention encompasses a process of contacting the surface of a substrate having biomolecules deposited thereupon with a basic, aqueous solution, followed by contacting the surface with a blocking agent that bonds preferentially to surface reactive moieties on the surface and reduces background signal and/or reduces the number of sites available for non-specific binding to occur. The invention also encompasses bioarrays fabricated using such process.

### FIELD OF THE INVENTION

The invention relates generally to biomolecules bound in array format to an insoluble substrate surface. More specifically, the invention relates to a method for modifying the surface of the bio-array to reduce background signal obtained in assays using the bio-array.

### BACKGROUND OF THE INVENTION

Biomolecules, such as peptides or oligonucleotides, immobilized on planar substrates are increasingly useful as diagnostic or screening tools. Such “bioarrays” include regions of usually different biomolecules arranged in a predetermined configuration on the substrate. These regions (sometimes referenced as “features” or “spots”) are positioned at respective locations (“addresses”) on the substrate. The arrays, when exposed to a sample, will exhibit an observed binding pattern. This binding pattern can be detected upon interrogating or imaging the array. Data obtained from the interrogation or imaging process are then analyzed to determine information about the sample. For example, all polynucleotide targets (e.g. DNA) in a sample can be labeled with a suitable label (such as a fluorescent compound) to provide a suitable binding signal during the interrogation or imaging process, and the labeled sample can then be assayed using an oligonucleotide array. Following exposure to the labeled sample, the fluorescence pattern on the array can be accurately observed. Assuming that the different sequence oligonucleotides were correctly deposited in accordance with the predetermined configuration, then the observed binding pattern will be indicative of the presence and/or concentration of one or more polynucleotide components of the sample.

Bioarrays can be fabricated by depositing previously obtained biomolecules onto a substrate surface, or by in situ synthesis of the biomolecule on the substrate surface. Glass slides are frequently used as a support medium when a transparent substrate is desired, although other materials may also be used for the substrate, such as silica or plastic. Biomolecules can be deposited directly onto an untreated substrate surface, but a surface treatment process is usually employed to provide more suitable surface characteristics. Examples of known surface treatment processes include coating the surface of glass slides with poly-L-lysine or an aminosilane prior to deposition of the biomolecule.

Surfaces that are positively charged, e.g. those with amine moieties exposed on the surface, have affinity for the negative backbone of polynucleotides. This property is of use in binding polynucleotides to the surface, e.g where the polynucleotides initially bound to the surface via the charge interaction are then crosslinked to the surface by exposure to UV light. Slides coated with poly-L-lysine have a surface that is both hydrophobic and positively charged. The hydrophobic character of the surface minimizes spreading of the printed spots, and the charge appears to help position the DNA on the surface in a way that makes cross-linking more efficient.

The sensitivity and dynamic range of bioarray assays are directly related to the amount of biomolecule that is deposited at each feature (or “spot”) on the bio-array. It is desirable for each feature of the array to be nearly homogenous (that is, to provide a substantially equal (+/−20%) level of biomolecule or binding signal across the entire surface of the feature). Such homogeneity of features leads to easier image analysis and more accurate interpretation of the data.

Assays using bioarrays depend considerably on quality and uniformity of feature deposition and low background signal due to non-specific binding to the substrate. One parameter in the fabrication of bioarrays is the composition of the solution used to deposit the biomolecules on the substrate surface. Salt/sodium citrate buffer (SSC) is widely used but is reported to give low binding efficiency and poor uniformity of features. It has been found that SSC supplemented with 50% dimethylsulfoxide (DMSO) gives somewhat improved results; however, DMSO is an undesirable solvent due to its toxicity. Diehl et al. (Nucleic Acids Research, 2001, 29(7):e38) obtained improved results using a solution of N,N,N-trimethylglycine in SSC.

Another aspect of bioarray fabrication is the processing of the substrate after feature deposition (after the biomolecule “spots” have been deposited on the substrate). Such processing is used to block unreacted binding sites on the substrate surface to reduce later non-specific binding. A typical method of blocking poly-L-lysine or other surfaces bearing amine groups involves contacting a bioarray substrate (having biomolecule features deposited thereupon) with a solution of succinic anhydride in aqueous, borate-buffered 1-methyl-2-pyrrolidinone (NMP). This acylation reaction converts free amine groups to carboxyl-terminated groups. Another known protocol (DeRisis et al. 1996 Nature Genetics 14:457-60) describes a surface treatment in which a poly-L-lysine coated surface is incubated in a series of solutions including a succinic anhydride solution, followed by boiling water and then ethanol. A typical problem that is observed when these protocols are followed is that the biomolecules that make up the features migrate across the substrate surface leading to an elongated “comet-tail” morphology of the features. When the features are closely spaced, this smearing can result in contamination of the biomolecule in a particular feature with the biomolecule from a nearby feature. Diehl et al. (Nucleic Acids Research, 2001, 29(7):e38) observed smearing of the array features using an aqueous acylation process; they reported better results were obtained using a non-aqueous dichloroethane solvent with succinic anhydride and N-methylimidazole to block nonspecific binding.

Further aspects of surface preparation prior to feature deposition may be found in the following references. U.S. Pat. No. 6,171,797 to Perbost teaches modifying the substrate surface to enable polynucleotides to bind to the surface via a cycloaddition reaction, e.g. through the reaction of a diene with a dienophile. U.S. Pat. No. 6,235,488 to Tom-Moy et al. describes the use of an organosilane compound to coat the surface of a sensor. Lenigk et al. teach binding of thiol-terminated polynucleotides to a silicon substrate surface using an organosilane. U.S. Pat. No. 6,258,454 to Lefkowitz et al. also describe functionalization of substrate surfaces with silane mixtures. Strother et al. (Nucleic Acids Research, 2000, 28(18):3535-41) teach functionalization of silicon surfaces with a protected aminoalkene followed by covalent attachment of polynucleotides to the amine-modified surface. Frutos et al. describes coating gold surfaces with monolayers of alkanethiols and forming DNA arrays using the modified surfaces. U.S. Pat. No. 5,919,523 to Sundberg et al. describes derivatization of solid supports for oligomer synthesis.

Beier et al. (Nucleic Acids Research, 1999, 27(9):1970-77) teach a process of preparing substrates for DNA arrays where the process includes washing the substrates in a 10% NaOH solution followed by an aminosilane solution. The surface is then activated for binding to polynucleotides. Beier et al. further teach that, following deposition of the polynucleotides on the surface using a basic aqueous solution, the surface is deactivated using a solution including diisopropylethylamine in dimethylformamide (DMF) and one of the following: 6-amino-1-hexanol, 1,3-diaminopropane, 3-amino-1-propanol, or 1 aminopropane. Brown et al. also teach a method of cleaning glass slides with a solution of NaOH in aqueous ethanol. This cleaning process is used prior to a treatment of the surface with poly-L-lysine. The biomolecules are then deposited on the poly-L-lysine coated glass slide to form the features of the array.

Miyachi et al. (Biotechnolog & Bioengineering, 2000, 69(3):323-29) reported making a DNA array having reduced non-specific binding compared to a conventional DNA array having a poly-L-lysine-modified substrate by modifying the substrate surface with a layer of hexamethyldisiloxane and streptavidin prior to depositing the DNA. Miyachi et al. further teach the use of a 50 mM NaOH solution for stripping sample DNA from the array for re-use of the array.

Hetero bifunctional cross-linkages have been used to couple 3′ or 5′-thio-modified oligonucleotides onto amino-propyl silanized surfaces as reported by Chrisey et al. (Nucleic Acids Res. 24: 3031-39 (1996)) and Guo et al. (Nucleic Acids Res. 22: 4556-5465 (1994)). U.S. Pat. No. 5,919,626 to Shi et al. describes the attachment of unmodified nucleic acids to silanized solid phase surfaces. The examples in Shi et al. '626 show the fabrication of DNA arrays through the use of inkjet printing or robotic micropipetting of solutions of DNA onto a substrate surface. U.S. Pat. No. 6,077,674 to Schleifer et al. describes a method of producing oligonucleotide arrays with features of high purity by modifying full length oligonucleotides with a linking group and binding the oligonucleotides to the array surface via the linking group, thus preventing shorter length oligonucleotides (resulting from ‘failures’ during the synthesis) from binding.

### SUMMARY OF THE INVENTION

The invention is thus addressed to the aforementioned deficiencies in the art, and provides novel methods for surface treatment of bioarrays to provide desirable substrate surface characteristics, and novel methods for fabrication of bioarrays. The present invention encompasses a process of contacting the surface of a substrate having biomolecules deposited thereupon with a basic solution, followed by contacting the surface with a blocking agent. The invention also encompasses bioarrays fabricated using such process.

In one aspect of the invention, a method is provided for treating the surface of the bioarray substrate to provide a modified substrate surface. The method includes contacting the bioarray with a basic solution and then contacting the bioarray with a blocking agent. In one embodiment the blocking agent is a cyclic anhydride, e.g. succinic anhydride. Prior to treatment according to the invention, the substrate presents reactive moieties on its surface. The reactive moieties provide sites that bind and react with biomolecules deposited on the surface. The reactive moieties displayed on the surface of the substrate typically are positively charged, e.g. amine groups, and give the substrate surface a net positive characteristic; the blocking agent modifies the moieties on the surface to alter the net positive characteristic. The result of the overall process is to alter the net surface charge of the substrate surface and/or to alter the moieties displayed on the surface. The surface of the substrate is preferably contacted with a basic aqueous solution after the deposition of the biomolecules on the surface of the substrate and before the substrate is contacted with a blocking agent.

Additional objects, advantages, and novel features of this invention shall be set forth in part in the descriptions and examples that follow and in part will become apparent to those skilled in the art upon examination of the following specifications or may be learned by the practice of the invention. The advantages of the invention may be realized and attained by means of the instruments, combinations, compositions and methods particularly pointed out in the appended claims.

### BRIEF DESCRIPTION OF THE DRAWINGS

These and other features of the invention will be understood from the description of representative embodiments of the method herein and the disclosure of illustrative apparatus for carrying out the method, taken together with the Figures, wherein

FIG. 1 is a flowchart schematically illustrating the method of the invention, as used in a larger process of fabricating a bioarray and using the bioarray in an assay.

FIG. 2 depicts a substrate bearing multiple bioarrays, as may be produced as described herein.

FIG. 3 is an enlarged view of a portion of FIG. 2 showing some of the identifiable individual regions (spots, or features) of a single bioarray of FIG. 2.

To facilitate understanding, identical reference numerals have been used, where practical, to designate corresponding elements that are common to the Figures. Figure components are not drawn to scale.

### DETAILED DESCRIPTION

Before the invention is described in detail, it is to be understood that unless otherwise indicated this invention is not limited to particular materials, reagents, reaction materials, manufacturing processes, or the like, as such may vary. It is also to be understood that the terminology used herein is for purposes of describing particular embodiments only, and is not intended to be limiting. It is also possible in the present invention that steps may be executed in different sequence where this is logically possible. However, the sequence described below is preferred.

It must be noted that, as used in the specification and the appended claims, the singular forms “a,” “an” and “the” include plural referents unless the context clearly dictates otherwise. Thus, for example, reference to “an insoluble support” includes a plurality of insoluble supports. In this specification and in the claims that follow, reference will be made to a number of terms that shall be defined to have the following meanings unless a contrary intention is apparent:

As used herein, polynucleotides include single or multiple stranded configurations, where one or more of the strands may or may not be completely aligned with another. The terms “polynucleotide” and “oligonucleotide” shall be generic to polydeoxynucleotides (containing 2-deoxy-D-ribose), to polyribonucleotides (containing D-ribose), to any other type of polynucleotide which is an N-glycoside of a purine or pyrimidine base, and to other polymers in which the conventional backbone has been replaced with a non-naturally occurring or synthetic backbone or in which one or more of the conventional bases has been replaced with a non-naturally occurring or synthetic base.

A “nucleotide” refers to a sub-unit of a nucleic acid (whether DNA or RNA or analogue thereof) which includes a phosphate group, a sugar group and a nitrogen containing base, as well as analogs of such sub-units. A “nucleoside” references a nucleic acid subunit including a sugar group and a nitrogen containing base. A “nucleoside moiety” refers to a molecule having a sugar group and a nitrogen containing base (as in a nucleoside) as a portion of a larger molecule, such as in a polynucleotide, oligonucleotide, or nucleoside phosphoramidite. A “nucleotide monomer” refers to a molecule which is not incorporated in a larger oligo- or poly-nucleotide chain and which corresponds to a single nucleotide sub-unit; nucleotide monomers may also have activating or protecting groups, if such groups are necessary for the intended use of the nucleotide monomer. A “polynucleotide intermediate” references a molecule occurring between steps in chemical synthesis of a polynucleotide, where the polynucleotide intermediate is subjected to further reactions to get the intended final product, e.g. a phosphite intermediate which is oxidized to a phosphate in a later step in the synthesis, or a protected polynucleotide which is then deprotected. An “oligonucleotide” generally refers to a nucleotide multimer of about 2 to 100 nucleotides in length, while a “polynucleotide” includes a nucleotide multimer having any number of nucleotides. It will be appreciated that, as used herein, the terms “nucleoside” and “nucleotide” will include those moieties which contain not only the naturally occurring purine and pyrimidine bases, e.g., adenine (A), thymine (T), cytosine (C), guanine (G), or uracil (U), but also modified purine and pyrimidine bases and other heterocyclic bases which have been modified (these moieties are sometimes referred to herein, collectively, as “purine and pyrimidine bases and analogs thereof”). Such modifications include, e.g., methylated purines or pyrimidines, acylated purines or pyrimidines, and the like, or the addition of a protecting group such as acetyl, difluoroacetyl, trifluoroacetyl, isobutyryl, benzoyl, or the like. The purine or pyrimidine base may also be an analog of the foregoing; suitable analogs will be known to those skilled in the art and are described in the pertinent texts and literature. Common analogs include, but are not limited to, 1-methyladenine, 2-methyladenine, N6-methyladenine, N6-isopentyladenine, 2-methylthio-N6-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromoguanine, 8-chloroguanine, 8-aminoguanine, 8-methylguanine, 8-thioguanine, 5-fluorouracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl)uracil, 5-(methylaminomethyl)uracil, 5-(carboxymethylaminomethyl)-uracil, 2-thiouracil, 5-methyl-2-thiouracil, 5-(2-bromovinyl)uracil, uracil-5-xyacetic acid, uracil-5-oxyacetic acid methyl ester, pseudouracil, 1-methylpseudouracil, queosine, inosine, 1-methylinosine, hypoxanthine, xanthine, 2-aminopurine, 6-hydroxyaminopurine, 6-thiopurine and 2,6-diaminopurine.

An “intemucleotide bond” refers to a chemical linkage between two nucleoside moieties, such as a phosphodiester linkage in nucleic acids found in nature, or such as linkages well known from the art of synthesis of nucleic acids and nucleic acid analogues. An internucleotide bond may comprise a phospho or phosphite group, and may include linkages where one or more oxygen atoms of the phospho or phosphite group are either modified with a substituent or replaced with another atom, e.g. a sulfur atom, or the nitrogen atom of a mono- or di-alkyl amino group. Such words as “bond,” “bound,” “binds,” or “binding,” may be used to express various modes of chemical binding, including covalent, ionic, hydrogen bonding, hydrophobic bonding, or mixed mode binding (combinations of the above); context may dictate when a specific meaning is permissible or required.

As used herein, the term “amino acid” is intended to include not only the L-, D- and nonchiral forms of naturally occurring amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine), but also modified amino acids, amino acid analogs, and other chemical compounds which can be incorporated in conventional oligopeptide synthesis, e.g., 4-nitrophenylalanine, isoglutamic acid, isoglutamine, ε-nicotinoyl-lysine, isonipecotic acid, tetrahydroisoquinoleic acid, α-aminoisobutyric acid, sarcosine, citrulline, cysteic acid, t-butylglycine, t-butylalanine, phenylglycine, cyclohexylalanine, β-alanine, 4-aminobutyric acid, and the like. An “oligopeptide” is a molecule containing from 2 to about 100 amino acid subunits. “Polypeptide” refers to a molecule having any number of amino acid subunits. “Biomolecule” refers to molecules generally derivable from living organisms, or analogues thereof. Biomolecules include, e.g. amino acids, oligopeptides, polypeptides, nucleotide monomers, oligonucleotides, polynucleotides, saccharides, polysaccharides, hormones, growth factors, peptidoglycans, or the like.

An “array”, unless a contrary intention appears, includes any one, two or three dimensional arrangement of addressable regions bearing a particular chemical moiety or moieties (for example, polynucleotide sequences) associated with that region. A “bioarray” is an array of biomolecules. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). In the case of an array, the “target” will be referenced as a moiety in a mobile phase (typically fluid), to be detected by probes (“target probes”) which are bound to the substrate at the various regions. However, either of the “target” or “target probes” may be the one which is to be evaluated by the other (thus, either one could be an unknown mixture of polynucleotides to be evaluated by binding with the other). While probes and targets of the present invention will typically be single-stranded, this is not essential. An “array layout” refers to one or more characteristics of the array, such as feature positioning, feature size, and some indication of a moiety at a given location. “Feature deposition” refers to a process of putting biomolecules on the substrate surface after the surface is prepared; feature deposition encompasses, e.g methods of in situ synthesis, placing droplets of biomolecules on the surface, and crosslinking of biomolecules to the surface. Reactive moieties are chemical groups on the substrate surface that provide sites that bind and react with biomolecules deposited on the surface. A “blocking agent” is one that bonds preferentially to surface reactive moieties and reduces background signal and/or reduces the number of sites available for non-specific binding to occur. Non-specific binding results from binding of sample (or “target”) molecules at sites other than the intended feature or unintended binding of sample molecules at a feature. “Passivation” refers to any process of chemically modifying the surface of a substrate, e.g. to block non-specific binding.

The term “alkyl” as used herein, unless otherwise specified, refers to a saturated straight chain, branched or cyclic hydrocarbon group of 1 to 24, typically 1-12, carbon atoms, such as methyl, ethyl, n-propyl, isopropyl, n-butyl, isobutyl, t-butyl, pentyl, cyclopentyl, isopentyl, neopentyl, hexyl, isohexyl, cyclohexyl, 3-methylpentyl, 2,2-dimethylbutyl, and 2,3-dimethylbutyl. The term “lower alkyl” intends an alkyl group of one to eight carbon atoms, and includes, for example, methyl, ethyl, n-propyl, isopropyl, n-butyl, isobutyl, t-butyl, pentyl, cyclopentyl, isopentyl, neopentyl, hexyl, isohexyl, cyclohexyl, 3-methylpentyl, 2,2-dimethylbutyl, and 2,3-dimethylbutyl. The term “cycloalkyl” refers to cyclic alkyl groups such as cyclopropyl, cyclobutyl, cyclopentyl, cyclohexyl, cycloheptyl and cyclooctyl. Alkyl groups include both substituted and unsubstituted alkyl groups. Typical substituents include one or more lower alkyl, any halogen, hydroxy, thio, or aryl, or optionally substituted on one or more available carbon atoms with a nonhydrocarbyl substituent such as cyano, nitro, halogen, hydroxyl, or the like.

It is known in the art applying to bioarray manufacture and use that non-specific binding of molecules to the surface of a bioarray may be reduced by treating the surface with a blocking agent. We have now discovered that this effect is improved upon by washing the bioarray surface with a basic solution prior to treatment with the blocking agent. The current invention may broadly be described as a method of treating a surface of a bioarray resulting in a modification of the surface that, if left unmodified, would result in excess noise or interference during use of the bioarray in conducting an assay. The treatment process of the current invention includes contacting the surface of the bioarray with a basic solution, followed by contacting the surface of the bioarray with a blocking agent. The contacting may be accomplished by, e.g. soaking the surface in a bath of the basic solution (or blocking agent), pouring a stream of basic solution (or blocking agent) across the surface, depositing the basic solution (or blocking agent) on specific portions of the surface through pipetting or other means, or any other method of bringing the surface into contact with the basic solution (or blocking agent).

The basic solution may be an aqueous basic solution or a non-aqueous basic solution. If aqueous, the solution should have a pH of at least about 9.0, but preferably greater than about 10, or greater than about 10.5, or greater than about 11.0, or greater than about 11.5. An aqueous solution with a pH of greater than about 13 may potentially lead to degradation of the biomolecules or of the surface (e.g. glass) if the duration of contact is too long, but this may be adjusted for with a shorter duration of contact. It lies within ordinary skill to adjust pH and duration of contact accordingly to achieve optimal results. Typically, the duration of washing (contacting the surface) with the basic solution will be at least about 1 second, preferably at least about 15 seconds, or at least about 30 seconds, 1 minute, or 2 minutes and may extend to as long as 30 minutes or more, but preferably less than about 20 minutes or more preferably less than about 10 minutes. Thus, contacting the bioarray surface with the basic solution is performed for a period of time and under conditions sufficient to reduce background signal of the assay using the bioarray, but short enough not to significantly degrade the biomolecules making up the feature.

An aqueous basic solution may be obtained by, e.g. dissolving a base in water or adjusting the pH of a solution of a buffer species by adding a base or an acid to the solution. The buffer species may be selected from those commonly used in chemical or biological techniques, e.g. carbonate, phosphate, tris, glycine, borate, tetraborate, diethanolamine, buffer species having pKa's in the desired range of 9-13, and the like. The base may also be selected from NaOH, KOH, or LiOH. Preferred concentration of the base in the aqueous basic solution ranges from 0.1 mM to 1.0 M. A solution of 50 mM NaOH in water has been found to be an effective choice of aqueous basic solution. In an embodiment, the aqueous basic solution includes both water and a water-miscible solvent, where at least 20% by volume is water, or preferably at least 50% by volume is water, or preferably at least 80% by volume is water. Preferred water-miscible solvents include methanol, ethanol, isopropanol, acetonitrile, and the like.

A non-aqueous basic solution may be used to contact the bioarray surface with a basic solution. The non-aqueous basic solution may be obtained by, e.g. dissolving a base in a non-aqueous solvent. Representative bases include alkylamines, e.g. triethlyamine, diisoproplyamine, or diisopropylethylamine; and other bases. Representative non-aqueous solvents include ethanol, dicloroethane, acetonitrile, dimethylformamide, tetrahydrofuran, dioxane, and the like, and two or more of the above in combination. A solution of 50 mM NaOH in methanol is one example of a non-aqueous basic solution. Alternatively, the non-aqueous basic solution may comprise an organic solvent that is itself basic, e.g. pyridine, lutidine, or piperidine, and may optionally have one or more bases dissolved in the organic solvent that is basic. Whether the basic solution is aqueous or non-aqueous, the composition of the basic solution should be selected to be compatible with the biomolecules on the array and with the succeeding treatment with the blocking agent.

The contacting of the bioarray surface with the basic solution is followed by contacting the surface with a blocking agent. The blocking agent is a reagent that participates in a reaction with surface reactive moieties of the bioarray to modify the surface, e.g. by changing the net surface charge or by changing the functionality of the reactive moiety. In particular embodiments, prior to treatment according to the present invention, the reactive moieties on the surface have positively charged groups (possibly via a process of surface modification to add positive reactive groups to the surface, e.g lysine moieties or aminosilane moieties). Where the bioarray surface presents amine moieties, any acylation reaction can be used. A preferred acylation reagent that may be used for a blocking agent is an anhydride, particularly a cyclic dicarboxylic acid (that is, an cyclic anhydride such as succinic anhydride, maleic anhydride, or glutaric anhydride), because once it has reacted with the amine on the surface, the product has a carboxylic acid group as an end functionality, which most likely will be negatively charged under hybridization conditions. This change in charge from positive to negative will tend to reduce non-specific binding of negatively charged molecules like polynucleotides. Alternate acylation reagents that result in a neutral group, thus altering the net positive charge of the surface towards neutral, may be used as the blocking agent. The blocking agent and the reaction conditions may be selected to optimize the blocking reaction to be much more specific to primary amine, alkyl amine and aliphatic amine, to avoid side reaction with the DNA (or other biomolecule) spotted in the features.

The method for treating the surface of the bioarray substrate provides a modified substrate surface. The result of the overall process is to alter the net surface charge of the substrate surface and/or to alter the moieties displayed on the surface. Initially, the moieties displayed on the surface of the substrate typically are positively charged and give the substrate surface a net positive characteristic; the blocking agent modifies the moieties on the surface to alter the net positive characteristic.

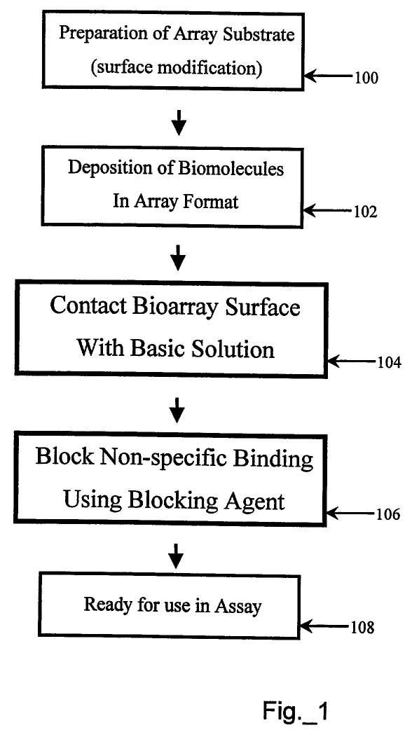

Referring now to FIG. 1, the present invention is shown in context of a portion of a process of fabricating a bioarray that is to be used in an assay. The preparation of the bioarray starts with preparation of an array substrate 100, including any desired surface modification, e.g. providing amine groups on the surface. Various methods such as inkjet printing or capillary deposition methods are then used to deposit biomolecules in an array format on the substrate surface 102. The method currently described is then performed on the bioarray substrate, starting with contacting the surface with a basic solution 104, followed by blocking non-specific binding by contacting the surface with a blocking agent 106. The bioarray is then ready for use in an assay 108.

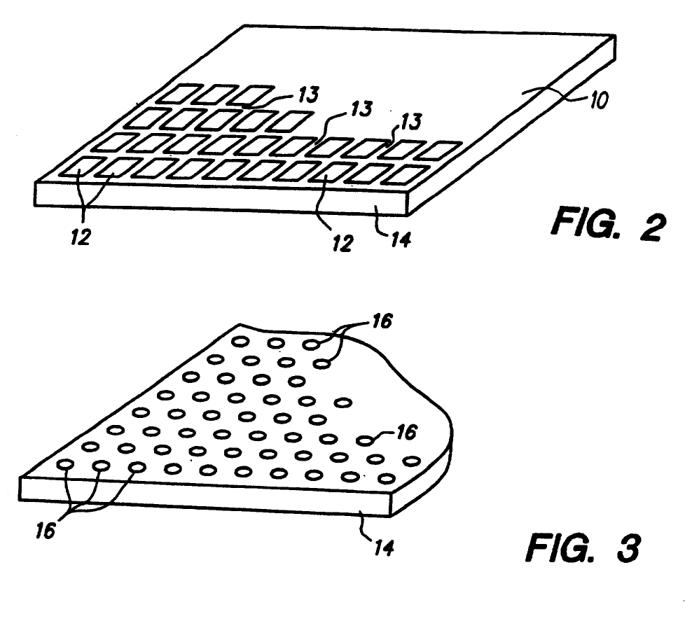

Referring now to FIGS. 2 and 3, the invention as described herein may be practiced to produce one or more arrays 12 (only some of which are shown in FIG. 2) across the surface of a single substrate 14. The arrays 12 produced on a given substrate need not be identical and some or all could be different. FIG. 3 depicts a single array 12 having multiple spots or features, 16. An array 12 may contain any number of multiple features, generally including at least tens of features, usually at least hundreds, more usually thousands, and as many as a hundred thousand or more features. All of the features 16 may be different, or some or all could be the same. Each region carries a predetermined biomolecule or a predetermined mixture of biomolecules, such as a particular polynucleotide sequence or a predetermined mixture of polynucleotides. The features of the array may be arranged in any desired pattern, e.g. organized rows and columns of features (for example, a grid of features across the substrate surface), a series of curvilinear rows across the substrate surface (for example, a series of concentric circles or semi-circles of features), and the like. In embodiments where very small feature sizes are desired, the density of features on the substrate may range from at least about ten features per square centimeter, or preferably at least about 35 features per square centimeter, or more preferably at least about 100 features per square centimeter, and up to about 1000 features per square centimeter, or preferably up to about 10,000 features per square centimeter, or perhaps up to 100,000 features per square centimeter. [* check if ranges are suitable *].

In one embodiment, about 10 to 100 of such arrays can be fabricated on a single substrate (such as glass). In such embodiment, after the substrate has the polynucleotides on its surface, the substrate may be cut into substrate segments, each of which may carry one or two arrays. It will also be appreciated that there need not be any space separating arrays from one another. Where a pattern of arrays is desired, any of a variety of geometries may be constructed, including for example, organized rows and columns of arrays (for example, a grid of arrays, across the substrate surface), a series of curvilinear rows across the substrate surface (for example, a series of concentric circles or semi-circles of arrays), and the like.

The array substrate may take any of a variety of configurations ranging from simple to complex. Thus, the substrate could have generally planar form, as for example a slide or plate configuration, such as a rectangular- or square- or disc-shape. In many embodiments, the substrate will be shaped generally as a rectangular solid, having a length in the range about 4 mm to 400 mm, usually about 4 mm to 150 mm, more usually about 4 mm to 125 mm; a width in the range about 4 mm to 400 mm, usually about 4 mm to 120 mm and more usually about 4 mm to 80 mm; and a thickness in the range about 0.01 mm to 5.0 mm, usually from about 0.1 mm to 2 mm and more usually from about 0.2 to 1 mm. The substrate surface onto which the polynucleotides are bound may be smooth or substantially planar, or have irregularities, such as depressions or elevations. The configuration of the array may be selected according to manufacturing, handling, and use considerations.

The process of the current invention may be employed on bioarrays fabricated on any substrate having a surface to which chemical entities may bind. Preferred substrate materials provide physical support for the deposited material and endure the conditions of the deposition process and of any subsequent treatment or handling or processing that may be encountered in the use of the particular array. Suitable substrates may have a variety of forms and compositions and may derive from naturally occurring materials, naturally occurring materials that have been synthetically modified, or synthetic materials. Examples of suitable substrate materials include, but are not limited to, nitrocellulose, glasses, silicas, teflons, and metals (for example, gold, platinum, and the like). Suitable substrate materials also include polymeric materials, including plastics (for example, polytetrafluoroethylene, polypropylene, polystyrene, polycarbonate, and blends thereof, and the like), polysaccharides such as agarose (e.g., that available commercially as Sepharose®, from Pharmacia) and dextran (e.g., those available commercially under the tradenames Sephadex® and Sephacyl®, also from Pharmacia), polyacrylamides, polystyrenes, polyvinyl alcohols, copolymers of hydroxyethyl methacrylate and methyl methacrylate, and the like.

The substrate surface may optionally exhibit surface modifications over a portion or over all of the surface with one or more different layers of compounds that serve to modify the properties of the surface in a desirable manner. Such modifications include: inorganic and organic layers such as metals, metal oxides, conformal silica or glass coatings, polymers, small organic molecules, hetero-bifunctional linking molecules, and the like. Polymeric layers of interest include layers of: polypeptides, proteins, polynucleotides or mimetics thereof, e.g. peptide nucleic acids and the like; polysaccharides, phospholipids, polyurethanes, polyesters, polycarbonates, polyureas, polyamides, polyethyleneamines, polyarylene sulfides, polysiloxanes, polyimides, polyacetates, and the like, where the polymers may be hetero- or homopolymeric, and may or may not have separate functional moieties attached thereto, e.g. conjugated.

In one embodiment, the bioarray has features comprising oligopeptides deposited on the surface of the substrate. In other embodiments, other biomolecules, such as polypeptides, oligonucleotides, polynucleotides, or known analogues or derivatives of any of the foregoing, or combinations of any of the foregoing. Any given feature can have the same or a different biomolecule or combination of biomolecules compared to any other given feature. Biomolecules may be derived from natural sources (e.g. isolated from cellular material) or may be synthetic. Examples of biomolecules include antigenic epitopes, fragments of antibodies or other proteins, polysacharrides, cDNAs, and RNAs.

In bioarray fabrication, the quantities of biomolecule available are usually very small and expensive. Additionally, sample quantities available for testing are usually also very small and it is therefore desirable to simultaneously test the same sample against a large number of different probes on an array. Therefore, one embodiment of the invention provides for fabrication of arrays with large numbers of very small, closely spaced features. Arrays may be fabricated with features that may have widths (that is, diameter, for a round spot) in the range from a minimum of about 10 micrometers to a maximum of about 1.0 cm. In embodiments where very small spot sizes or feature sizes are desired, material can be deposited in small spots whose width is in the range about 1.0 micrometer to 1.0 mm, usually about 5.0 micrometers to 0.5 mm, and more usually about 10 micrometers to 200 micrometers. Interfeature areas will typically (but not essentially) be present which do not carry any polynucleotide. It will be appreciated though, that the interfeature areas could be of various sizes and configurations.

The biomolecule is typically applied to the surface of the substrate by spotting, using pipettes, pins, inkjets, or the like. Methods of depositing materials onto a planar substrate include loading and then touching a pin or capillary to a surface, such as described in U.S. Pat. No. 5,807,522 to Brown et al. U.S. Pat. No. 6,110,426 to Shalon, et al. describes a method of dispensing a known volume of a reagent at each selected array position, by tapping a capillary dispenser on the substrate under conditions effective to draw a defined volume of liquid onto the substrate. Another method employs an array of pins dipped into corresponding wells, e.g., the 96 wells of a microtitre plate, for transferring an array of droplets to a substrate, such as a porous membrane. One such array of pins is designed to spot a membrane in a staggered fashion, for creating an array of 9216 spots in a 22 by 22 cm area (Lehrach, et al., “Hybrididization Fingerprinting in Genome Mapping and Sequencing,” in Genome Analysis, Vol. 1, pp. 39-81 (1990, Davies and Tilgham, Eds., Cold Spring Harbor Press)). A different method has been described which uses a vacuum manifold to transfer a plurality, e.g., 96, of aqueous samples of DNA from 3 millimeter diameter wells to a porous membrane for making ordered arrays of DNA on a porous membrane, i.e. a “dot blot” approach. A common variant of this procedure is a “slot-blot” method in which the wells have highly-elongated oval shapes. A method of making an oligonucleotide matrix by spotting DNA onto a thin layer of polyacrylamide has been described (Khrapko, et al. (1991) DNA Sequence 1:375-388). The spotting is done manually with a micropipette.

Ink jet technology may be used to spot biomolecules and other reagents on a surface, for example, using a pulse jet such as an inkjet type head to deposit a droplet of reagent solution for each feature. Such a technique has been described, for example, in PCT publications WO 89/10977, WO 95/25116 and WO 98/41531, and elsewhere. In such methods, the head has at least one jet which can dispense droplets of a fluid onto a substrate, the jet including a chamber with an orifice, and including an ejector which, when activated, causes a droplet to be ejected from the orifice. The head may particularly be of a type commonly used in inkjet printers, in which a plurality of pulse jets (such as those with thermal or piezoelectric ejectors) are used, with their orifices on a common front surface of the head. The head is positioned with the orifice facing the substrate. Multiple fluid droplets (where the fluid comprises the biomolecule) are dispensed from the head orifice so as to form an array of droplets on the substrate (this formed array may or may not be the same as the final desired array since, for example, multiple heads can be used to form the final array and multiple passes of the head(s) may be required to complete the array).

As is well known in the ink jet print art, the amount of fluid that is expelled in a single activation event of a pulse jet, can be controlled by changing one or more of a number of parameters, including the orifice diameter, the orifice length (thickness of the orifice member at the orifice), the size of the deposition chamber, and the size of the heating element, among others. The amount of fluid that is expelled during a single activation event is generally in the range about 0.1 to 1000 pL, usually about 0.5 to 500 pL and more usually about 1.0 to 250 pL. A typical velocity at which the fluid is expelled from the chamber is more than about 1 m/s, usually more than about 10 m/s, and may be as great as about 20 m/s or greater. As will be appreciated, if the orifice is in motion with respect to the receiving surface at the time an ejector is activated, the actual site of deposition of the material will not be the location that is at the moment of activation in a line-of-sight relation to the orifice, but will be a location that is predictable for the given distances and velocities.

Still other methods and apparatus for fabrication of polynucleotide arrays are described in, e.g. U.S. Pat. No. 6,242,266 to Schleiffer et al., which describes a fluid dispensing head for dispensing droplets onto a substrate, and methods of positioning the head in relation to the substrate. Other methods include those disclosed by U.S. Pat. No. 6,180,351 to Cattell; U.S. Pat. No. 6,171,797 to Perbost; Gamble, et al., WO97/44134; Gamble, et al., WO98/10858; Baldeschwieler, et al., WO95/25116; and the like.

A number of other known methods are available and may be used for depositing the biomolecules on the substrate surface. Modifications of these known methods within the capabilities of a skilled practitioner in the art as well as other methods known to those of skill in the art may be employed. It should be specifically understood that, in addition to inkjet methods, other methods can also be used to deposit biomolecules on the substrate surface, including those such as described in U.S. Pat. No. 5,807,522, or apparatus which may employ photolithographic techniques for forming arrays of moieties, such as described in U.S. Pat. No. 5,143,854 and U.S. Pat. No. 5,405,783, or any other suitable apparatus which can be used for fabricating arrays of moieties. For example, robotic devices for precisely depositing volumes of solutions onto discrete locations of a support surface, i.e. arrayers, are commercially available from a number of vendors, including: Genetic Microsystems; Cartesian Technologies; Beecher Instruments; Genomic Solutions; and BioRobotics. For further methods, see U.S. Pat. No. 5,143,854 to Pirrung et al.; Fodor et al., Science 251:767-773 (1991); Southern, et al. Genomics 13:1008-1017 (1992); PCT patent publications WO 90/15070 and 92/10092; U.S. Pat. No. 4,877,745 to Hayes et al.; U.S. Pat. No. 5,338,688 to Deeg et al.; U.S. Pat. No. 5,474,796 to Brennan; U.S. Pat. No. 5,449,754 to Nishioka; U.S. Pat. No. 5,658,802 to Hayes et al.; and U.S. Pat. No. 5,700,637 to Southern. Another strategy for forming bioarrays is discussed in U.S. Pat. No. 5,744,305 to Fodor, et al. and involves solid phase chemistry, photolabile protecting groups and photolithography. Still other patents and patent applications describing arrays of biopolymeric compounds and methods for their fabrication include: U.S. Pat. Nos. 5,242,974; 5,384,261; 5,412,087; 5,424,186; 5,429,807; 5,436,327; 5,445,934; 5,472,672; 5,527,681; 5,529,756; 5,545,531; 5,554,501; 5,556,752; 5,561,071; 5,599,695; 5,624,711; 5,639,603; 5,658,734; WO 93/17126; WO 95/11995; WO 95/35505; EP 742 287; and EP 799 897. Also of interest are WO 97/14706 and WO 98/30575.

The biomolecule may bind directly to the substrate surface or may bind via an intermediate moiety upon the surface, e.g. a bifunctional linker molecule or other surface treatment. Polynucleotides may be bound to the surface by irradiating with UV light, during which the polynucleotides covalently attach to the surface, typically via an intermediate moiety, presumably, by non-specific, free-radical cross-linking. Usually, the surface is irradiated with light, such as UV light, for a period of time that allows an energy transfer from about 200 millijoule to about 20000 millijoule, usually, from about 4500 to about 18000 millijoule, at a wavelength of from about 190 nm to about 400 nm, usually, from about 200 nm to about 300 nm. Chemical methods for covalently binding biomolecules in an array format to substrate surfaces are known in the art and may be employed by one of ordinary skill in the art.

### EXAMPLES

The practice of the present invention will employ, unless otherwise indicated, conventional techniques of organic chemistry, biochemistry, molecular biology, and the like, which are within the skill of the art. Such techniques are explained fully in the literature.

The following examples are put forth so as to provide those of ordinary skill in the art with a complete disclosure and description of how to perform the methods and use the compositions disclosed and claimed herein. Efforts have been made to ensure accuracy with respect to numbers (e.g., amounts, temperature, etc.) but some errors and deviations should be accounted for. Unless indicated otherwise, parts are parts by weight, temperature is at or near room temperature or is given in ° C., and pressure is at or near atmospheric. Standard temperature and pressure are defined as 20° C. and 1 atmosphere.

Preparation of poly-L-lysine Coated Slides

Glass slides are cleaned by soaking for two hours in a solution of 70 g NaOH dissolved in 280 ml ddH20+420 ml 95% ethanol. Slides are rinsed thoroughly in ddH20 and then coated by with poly-L-lysine by soaking for 15-60 minutes in a solution of 70 ml poly-L-lysine (Sigma) in 70 ml phosphate buffered saline (PBS) plus 560 ml ddH20. Slides are again rinsed in ddH2O, dried, and aged for a period of two weeks under clean, dry conditions.

Preparation of Glass Slides With Reactive Amine Surface

Clean glass microscope slides were washed and placed in a Teflon® slide holder. Anhydrous toluene (800 ml) was placed in a 2-liter reaction vessel into which the slide holder had been placed. The toluene was stirred vigorously. To the stirring toluene was added 34.4 g (0.156 moles) of 3-aminopropyltriethoxysilane (Aldrich Chemical Company). The reaction was allowed to proceed with stirring for one hour at room temperature. The glass slides were removed from the reaction vessel and were rinsed with anhydrous toluene. The slides were then rinsed successively with acetone and 2-propanol and then were dried with a stream of nitrogen. The dry slides were placed in a vacuum oven and dried at 150° C. at about 1 mm Hg for one hour.

Preparation of Glass Slides With High Density, Hydrophilic, Reactive Amine Surface

Clean glass microscope slides were washed and placed in a Teflon® slide holder. The holder containing the slides was placed into a 2-liter reaction vessel and purged with nitrogen for 1 hour. In a 1000 ml oven-dried bottle capped with a septum, 800 ml of anhydrous toluene was cannulated and 10 g of 11-Bromoundecyl trichlorosilane (from Gelest Inc., Tullytown, Pa.) was added via a syringe. The silane solution was swirled to dissolve the contents and transferred to the reactor via a cannula. The silylation was carried out at room temperature for 2 hours under nitrogen atmosphere. The silane solution was then removed from the reactor and replaced with 800 ml of fresh anhydrous toluene. After 5 minutes of stirring, the boat containing the silylated slides was removed from the reactor and put into a fresh toluene wash. The holder was washed thoroughly in the toluene. The toluene was removed and the slides were then washed with 800 ml of acetonitrile, then cured for 1 hour at 150° C. in a vacuum oven at about 1 mm Hg. The slides were dried with a stream of nitrogen and stored in a dry box prior to final conversion with 4,7,10-Trioxa-1,13-tridecanediamine (from Aldrich Chemical Company). The silylated slides obtained as described above were placed into a 2-liter reactor. The reactor was filled with 800 ml of 4,7,10-Trioxa-1,13-tridecanediamine and warmed to 100° C. The reaction was maintained at 100° C. overnight. The next morning the reaction was cooled to room temperature, the slides were removed from the reactor and were washed in a beaker successively with dimethylformamide (twice), acetonitrile (twice), by dipping the boat continuously up and down in the solvent wash. The slides were dried with a stream of nitrogen, wrapped in aluminum foil and stored in a dry box.

A robotic spotter was used to deposit arrays of polynucleotides on slides prepared as described above. The polynucleotides were crosslinked to the surface using UV light. The slides were then incubated for an hour with succinic anhydride (the blocking agent) in borate buffered 1-methyl-2-pyrolidinone according to a published protocol (Eisen et al., Meth. Enzymol (1999) 303: 179-205). The incubation with succinic anhydride was either preceded by washing the slides with 50 mM NaOH in water (basic solution) or the wash with the basic solution was skipped. Results were compared, and slides that had been washed with the basic solution preceding the blocking showed less smearing of the features. Assays of sample material performed with the slides confirmed that less non-specific binding was observed with the slides that had been washed with the basic solution.

While the foregoing embodiments of the invention have been set forth in considerable detail for the purpose of making a complete disclosure of the invention, it will be apparent to those of skill in the art that numerous changes may be made in such details without departing from the spirit and the principles of the invention. Accordingly, the invention should be limited only by the following claims.

All patents, patent applications, and publications mentioned herein are hereby incorporated by reference in their entireties.

What is claimed is:

1. A method of treating a surface of a bioarray having biomolecules deposited thereupon, the method comprising contacting the surface of the bioarray with a basic solution, and then contacting the surface of the bioarray with a blocking agent.

2. The method of claim 1, wherein the basic solution comprises at least 20% water and has a pH greater than 9.

3. The method of claim 1, wherein the basic solution is applied for less than about five minutes.

4. The method of claim 1, wherein the surface is positively charged due to positively charged reactive moieties bound to the surface.

5. The method of claim 1, wherein the blocking agent is an anhydride.

6. The method of claim 5, wherein the blocking agent is a cyclic anhydride.

7. The method of claim 6, wherein the cyclic anhydride is selected from the group consisting of succinic anhydride, maleic anhydride, and glutaric anhydride.

8. The method of claim 1, wherein the biomolecules are bound on the surface via a reactive moiety that is bound to the surface, wherein the reactive moiety can react to covalently bind the biomolecule to the surface.

9. The method of claim 8, wherein the reactive moiety comprises an amine group.

10. The method of claim 1, wherein the biomolecules are selected from the group consisting of polypeptides, polynucleotides, and polysaccharides.

11. A method of fabricating an array of biomolecules bound on a substrate surface, the method comprising obtaining a substrate having reactive moieties present thereupon, depositing biomolecules onto the substrate surface, wherein at least some biomolecules are bound to at least some of the reactive moieties present on the substrate, contacting the substrate surface with a basic solution, and contacting the substrate surface with a blocking agent to block at least some of the reactive moieties to yield the array.

12. The method of claim 11, wherein the biomolecules are synthesized in situ on the substrate surface.

13. The method of claim 11, wherein the basic solution comprises at least 20% water and has a pH greater than 9.

14. The method of claim 11, wherein the basic solution is applied for less than about five minutes.

15. The method of claim 11, wherein the surface is positively charged due to positively charged reactive moieties bound to the surface.

16. The method of claim 11, wherein the blocking agent is an anhydride.

17. The method of claim 16, wherein the blocking agent is a cyclic anhydride.

18. The method of claim 17, wherein the cyclic anhydride is selected from the group consisting of succinic anhydride, maleic anhydride, and glutaric anhydride.

19. The method of claim 11, wherein the reactive moieties comprises amine groups.

20. The method of claim 11 wherein the biomolecules are selected from the group consisting of polypeptides, polynucleotides, and polysaccharides.

21. In a method of fabricating a bioarray upon a substrate having a surface, the method comprising:
depositing biomolecules on the surface; and
contacting the surface with a blocking agent;
the inprovement comprising:
after depositing the molecules on the surface and before contacting the surface with the blocking agent, contacting the surface with a basic solution.

22. The improved method of claim 21, wherein the basic solution comprises at least 20% water and has a pH greater than 9.

23. The improved method of claim 21, wherein the basic solution is applied for less than about five minutes.

24. The improved method of claim 21, wherein the surface is positively charged due to positively charged reactive moieties bound to the surface.

25. The improved method of claim 21, wherein the blocking agent is an acylating agent selected from succinic anhydride, maleic anhydride, and glutaric anhydride.

26. The improved method of claim 17, wherein the biomolecules are selected from the group consisting of polypeptides, polynucleotides, and polysaccharides.
